(19) United States
(12) Patent Application Publication
(10) Pub. No.: US 2006/0183025 A1
(43) Pub. Date: Aug. 17, 2006

(54) Methods of forming mask patterns, methods of correcting feature dimension variation, microlithography methods, recording medium and electron beam exposure system

(75) Inventors: Baorui Yang, Boise, ID (US); Randal W. Chance, Boise, ID (US)

Correspondence Address: WELLS ST. JOHN P.S., 601 W. FIRST AVENUE, SUITE 1300, SPOKANE, WA, 99201, US

(73) Assignee: Micron Technology, Inc.

(21) Appl. No.: 11/058,553

(22) Filed: Feb. 14, 2005

(51) Int. Cl. G03F 1/00; G03C 5/00; G21G 5/00; A61N 5/00

(52) U.S. Cl. 430/5; 430/296; 430/942; 250/492.3; 430/30

(57) ABSTRACT

The invention includes methods of forming reticles. A mask blank is provided having a plurality of regions defined within a main-field area. Exposure to an electron beam is initiated at an initial locus within an interior region of the main-field. The invention includes a method of correcting feature dimension variation. A mask blank is patterned utilizing a first dose correction component and feature dimension variance is determined. The variance is utilized to determine a second correction component which is added to the first dose correction component to create an enhanced dose correction. The invention includes a recording medium and a system comprising the recording medium. The medium contains programming configured to cause processing circuitry to: access data defining a design pattern; obtain error data pertaining to feature dimension variation; generate correction data; produce data defining a corrective pattern; and apply the corrective pattern during an exposure event.

### TECHNICAL FIELD

The invention pertains to methods of forming mask patterns, methods of correcting feature dimension variation, microlithography methods, recording medium and microlithography exposure systems.

### BACKGROUND OF THE INVENTION

Radiation patterning tools are utilized to pattern radiation during, for example, semiconductor processing. The patterned radiation (such as, for example, UV light) is projected against a radiation-imagable material such as, for example, photoresist and utilized to create a pattern in the radiation-imagable material. The utilization of a patterned radiation for forming a desired pattern in a radiation-imagable material is typically referred to as photolithography. The radiation-patterning tool can be referred to as a photomask or reticle. The term “photomask” is traditionally understood to refer to masks which define a pattern for an entirety of a wafer, and the term “reticle” is traditionally understood to refer to a patterning tool which defines a patterned for only a portion of a wafer. However, the term “photomask”, or more generally “mask”, and “reticle” are frequently used interchangeably in modern parlance so that either term can refer to a radiation patterning tool that encompasses either a portion or an entirety of a wafer. For purposes of interpreting this disclosure and the claims that follow, the term “reticle” is utilized to generally refer to any radiation-patterning tool regardless of whether the tool is utilized to pattern an entirety of a substrate or only a portion of the substrate.

Reticles are typically manufactured by methods comprising microlithography techniques performed utilizing high energy tools which “write” a pattern onto a mask blank by exposing the mask blank to an exposure beam (e.g. an electron beam) in a predetermined write pattern. A particular write pattern is developed based upon a desired pattern for the final reticle. Typically, the pattern will comprise a plurality of pattern features. Data pertaining to the desired pattern, typically in a binary digital format, is fragmented or divided into data stripes which can be utilized to provide to an electron beam (e-beam) tool to write or expose portions of the mask blank. Each data stripe is written by scanning the exposure beam across the reticle blank in a series of frames such that the exposure defines the features of the mask.

Exposure of a mask blank (alternatively referred to as a reticle preform) to an incident electron beam is shown in FIG. 1. An electron beam lithography apparatus 10 can be utilized to form a feature pattern by providing a mask blank 12 within the apparatus and exposing the blank to an incident electron beam 18. Mask blank 12 can typically comprise a resist or other imagable material 16 over a substrate 14. A desired feature pattern is “written” onto the resist or imagable material by projecting the electron beam to expose select regions of the resist layer in accordance with pattern data. The exposed resist is then developed to produce the feature pattern within the resist layer. The pattern can then be transferred to one or more underlying-layers and the resist can ultimately be removed from the reticle structure.

During electron beam exposure, electrons from the electron beam can unintentionally expose portions of resist layer 16 other than, or in addition to, the intended portions. Additional and/or unintended exposure can result in variation of feature dimension or critical dimension (CD). The variance in critical dimension can be non-uniform throughout the pattern such that some feature dimensions in a particular area of the pattern are closer to the intended feature dimension than those in other areas of the pattern.

One cause of unintentional exposure which can result in feature dimension variation is illustrated in FIG. 1. As shown, an incident beam 18 is directed at a particular locus on the resist layer. Typically, the beam will be directed substantially perpendicular to the upper surface of the reticle preform. However, electrons from the incident beam can be scattered from a surface of the resist material from within the resist material and from underlying layers. These scattered electrons can be “re-scattered” or “backscattered” due to reflection from an inner surface of the resist layer or from reflection from the bottom of optical column 20. As shown in FIG. 1, re-scattered electrons 22 impinge upon unintended regions 15 of the resist layer. This unintentional exposure due to backscattering can change the critical dimension of an intended pattern producing a “re-scattering” or fogging effect.

Patterning methodology has been developed which attempts to correct the fogging effect by, for example, controlling or adjusting exposure dose. Typically, the correction is based upon an assumption that the electron fogging effect is a Gaussian distribution and thus the correction is symmetrical around the center of a patterned layout with more dose correction applied in the pattern center than near the edge or corner. A typical electron fogging effect correction on a mask is shown in FIG. 2. In the depicted symmetrical electron fogging correction the bright circle in the center corresponds to the correction for the main-field.

Although the symmetrical correction model is somewhat effective for correcting CD variations, such fails to account for additional factors that may contribute toward the fogging effect and thereby fails to produce global CD uniformity throughout the pattern. Accordingly, it would be desirable to develop alternative methodology for correction of fogging effects.

### SUMMARY OF THE INVENTION

In one aspect the invention encompasses a method of forming a mask pattern. A mask blank or “reticle preform” is provided which has a metallic layer disposed between a transparent substrate material and an imageable material. The mask blank has an upper surface comprising a main-field area bounded by a lateral periphery. A plurality of regions is defined within the main-field. The plurality of regions includes an inner region and multiple outer regions where the inner region is spaced from the entirety of the lateral periphery by at least one outer region. Exposure of the mask blank to an electron beam is initiated at an initial locus within the inner region.

In one aspect the invention encompasses a method of correcting feature dimension variation. A mask blank is exposed in accordance with an initial corrected write pattern which has a first dose correction component that is symmetrical. The initially exposed substrate is divided into a plurality of regions across and upper mask surface. The feature dimension is measured within each region and a variance is determined between the measured feature dimension and an intended feature dimension. The variance is utilized to determine a second dose correction component which is non-symmetrical. An enhanced dose correction is created which comprises the second dose correction component added to the first dose correction component which can achieve at least partial correction of the variance. The enhanced dose correction is applied to a write pattern to generate an enhanced correction write pattern and a subsequent mask blank is exposed in accordance with the enhanced dose correction write pattern.

In one aspect the invention encompasses a recording medium comprising programming configured to cause processing circuitry to perform processing. The processing includes accessing data defining a design pattern to be written onto a resist material by exposing to an exposure beam. The processing additionally includes obtaining error data pertaining to feature dimension variation caused by exposure beam deflection during writing of the design pattern. The processing additionally includes generating correction data based upon the error data, producing data defining a corrective pattern by adjusting the data defining the design pattern utilizing the corrected data, and applying the corrected pattern during an exposure event.

In one aspect the invention encompasses an electron beam exposure system. The system includes an electron beam source and a movable stage for supporting a substrate. The system additionally includes a processor which contains programming configured to cause processing circuitry to access exposure data, divide the exposure pattern into regions, determine a dose correction for each region to alleviate backscattering effects and alleviate beam deflection effects, and generate a corrected exposure pattern to apply a corrected dose for each region. The system additionally includes a controller which is configured to direct electrons emitted by the electron beam source to expose the layer of resist on a mask blank in accordance with the corrected exposure pattern.

### BRIEF DESCRIPTION OF THE DRAWINGS

The patent or application file contains at least one drawing executed in color. Copies of this patent or patent application publication with color drawings will be provided by the Office upon request and payment of the necessary fee.

Preferred embodiments of the invention are described below with reference to the following accompanying drawings.

FIG. 1 is a diagrammatic view of a lithography system illustrating a backscattering effect.

FIG. 2 illustrates a symmetrical electron fogging effect correction distribution.

FIG. 3 is a diagrammatic cross-sectional view of a reticle preform construction which can be processed in accordance with methodology of the present invention.

FIG. 4 is a diagrammatic view of a surface of the reticle preform construction shown in FIG. 3, with the cross-section of FIG. 3 being taken along line 3-3 of FIG. 4.

FIG. 5 is a diagrammatic illustration of a lithography system according to one aspect of the invention.

FIG. 6 schematically depicts a particular exposure ordering of sub-regions within a main-field.

FIG. 7 shows a critical dimension variance (or uniformity) map of a 90 nm design rule gate layer reticle with a writing order as shown in FIG. 6.

FIG. 8 shows an alternative scan order for exposing sub-regions within a main-field relative to that shown in FIG. 6.

FIG. 9 shows a critical dimension variance map of a 90 nm design rule gate layer reticle utilizing the scan order shown in FIG. 8.

FIG. 10 shows an alternative scan order of exposing areas within a field in accordance with one aspect of the invention.

FIG. 11 shows a critical dimension variance map of a 90 nm design rule gate layer reticle utilizing the writing order as shown in FIG. 10.

FIG. 12 is a schematic diagram of main deflection fields and stage moving directions. Panel A illustrates a main deflection field area having equivalent horizontal (x) and vertical (y) field dimensions. Panel B illustrates an alternative main deflection field obtained by alternative data fragmentation in accordance with one aspect of the invention.

FIG. 13 is a critical dimension uniformity map of a 90 nm design rule gate layer reticle having a frame height of 512 microns and having a write order in accordance with FIG. 6.

FIG. 14 illustrates a critical dimension uniformity map of a 90 nm design rule gate layer reticle utilizing a frame height of 768 microns and a write order in accordance with FIG. 6.

FIG. 15 is a flowchart for performing methodology in accordance with one aspect of the invention.

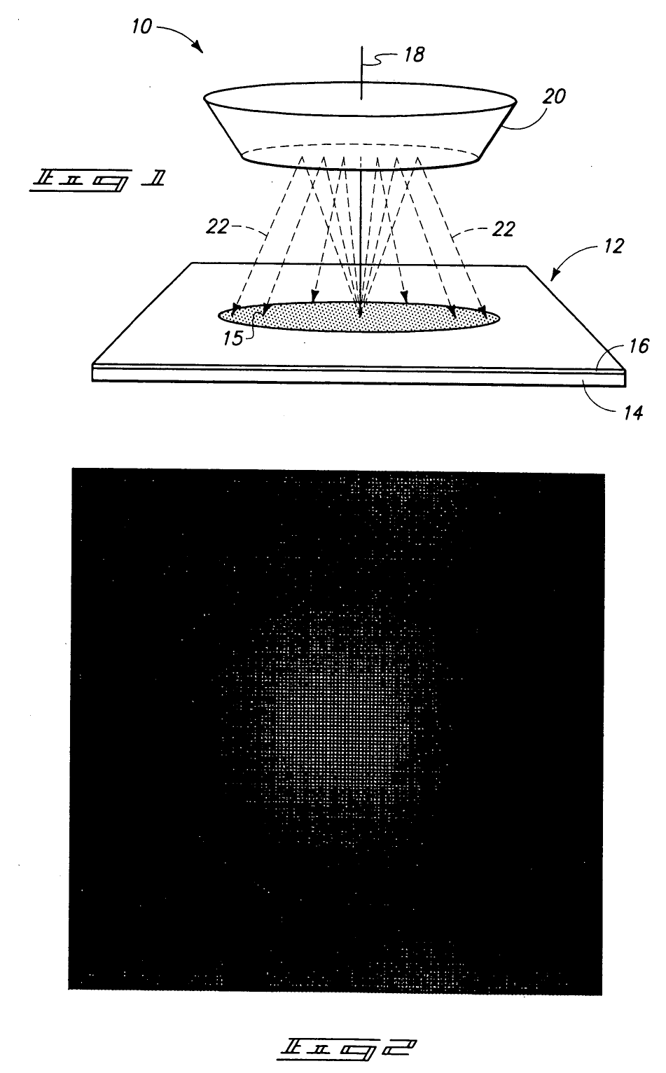

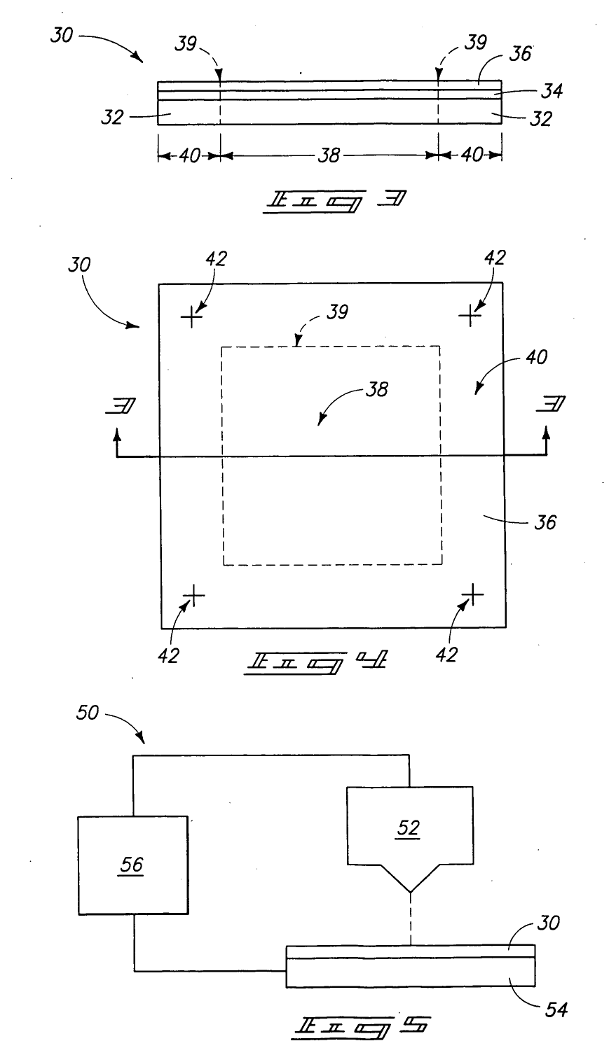

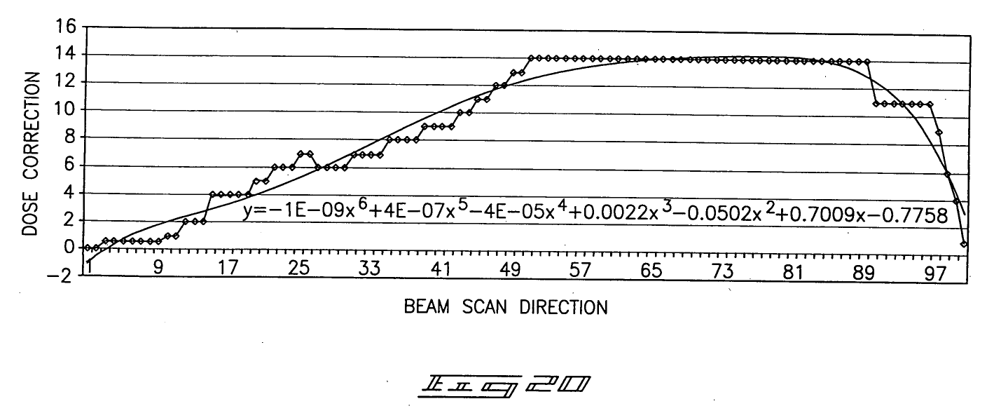

FIG. 14 depicts an exemplary lithography system which can be utilized for performing methodology in accordance with one aspect of the invention.

FIG. 17 is a functional block diagram of a processor device in accordance with one aspect of the invention.

FIG. 18 depicts a two component correction model in accordance with one aspect of the invention.

FIG. 19 shows an exemplary secondary correction component in accordance with on aspect of the invention.

FIG. 20 shows an alternative exemplary secondary correction component in accordance relative to that shown in FIG. 19.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The present invention relates to methodology and systems for correcting feature dimension variation which can occur during exposing a resist to form a feature pattern. In particular aspects, the invention pertains to correcting or alleviating feature dimension variation during electron beam lithography. It is to be understood however, that the techniques and methodology described herein can be adapted for application to other lithography techniques. Further, although the invention is described in terms of forming a mask or reticle the invention additionally contemplates utilization of the described aspects of the invention during alternative patterning application such as, for example, patterning a semiconductive wafer.

As discussed in the background section of this description, electron backscattering can create or contribute to an electron fogging effect resulting in variation of dimensions of features in a resulting pattern. The variation in feature dimension can be non-uniform throughout the pattern. One aspect of the present invention is a recognition that additional factors can contribute to the fogging effect. Specifically, as further discussed below, it is recognized that electron beam deflection and/or order of exposing areas within the pattern (write order or scan sequence) can contribute to the fogging effect. It is additionally recognized that such contribution can typically be non-symmetrical with respect to a center point of the pattern, at least in instances where a conventional scan sequence is utilized. It is also recognized that conventional symmetrical correction models only partially alleviate fogging effects and typically result in a mask or reticle having some degree of critical dimension variation. Particularly, where a conventional scan order and symmetrical correction model is utilized a resulting pattern can typically have a feature dimension variance with such variance being non-symmetrical relative to a center of the pattern.

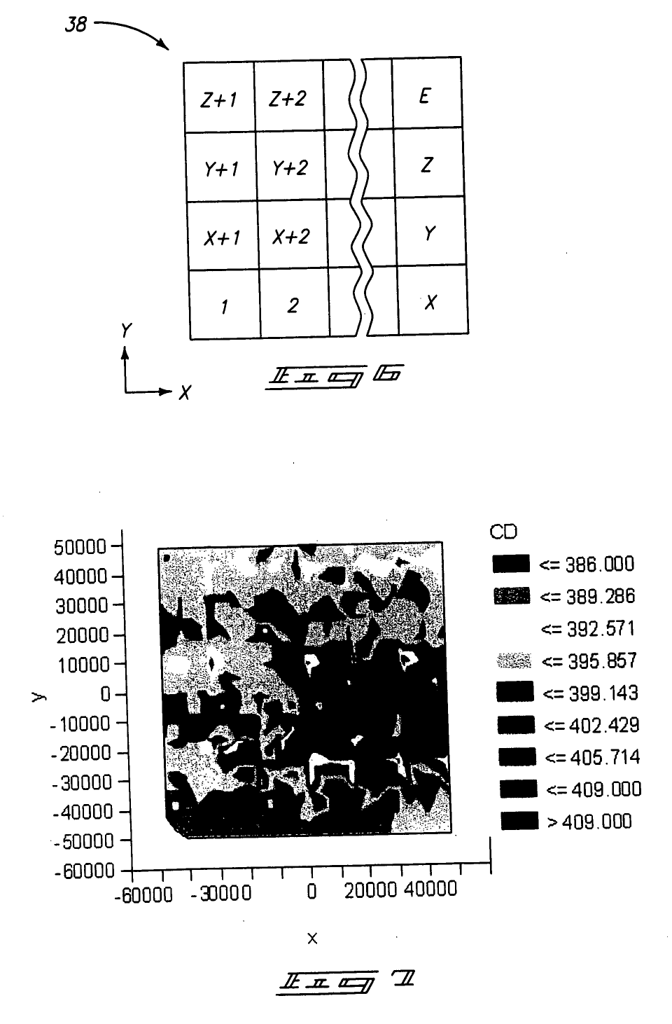

Exemplary methods of forming a reticle in accordance with an aspect of the present invention are described with reference to FIGS. 3-20. Referring initially to FIG. 3 such shows a reticle preform 30 (alternatively referred to as a mask blank), prior to any exposure to a processing beam. Preform 30 comprises a substrate 32 which is relatively transparent and an opaque layer 34 over the relatively transparent substrate. The term “relatively” is utilized to indicate that a material has a particular quantitative property relative to another. For instance the term “relatively opaque” is utilized to indicate that a material is more opaque than another material, with such other material being referred to as a “relatively transparent” material.

The relatively transparent material 32 will typically comprise, consist essentially of, or consist of quartz. The relatively opaque material 34 will typically be a metallic layer and can in particular applications comprise, consist essentially of, or consist of chromium. Although FIG. 3 depicts layer 34 as a homogenous layer, it is to be understood that in particular instances layer 34 can comprise two or more materials to form a composite layer. For example, layer 34 can comprise a layer of chromium over a layer of a phase-shifting material. Exemplary phase-shifting materials can be, for example, silicon nitride, silicon oxynitride, a metal silicide (i.e. molybdenum silicide), and/or Mo_{w}SiₓN_{y}O_{z} where w, x, y and z are numbers greater than zero. Reticles comprising one or more phase shift materials can typically be referred to as phase shift reticles. Where layer 34 lacks phase-shifting materials, for example, where layer 34 consists essentially of or consists of chromium, the patterned reticle can be referred to as a binary reticle.

A layer of resist or other imageable material 36 is present over opaque material 34. For purposes of the present description, the term ‘resist’ can refer to a material that is sensitive to irradiation such that its chemical properties change when irradiated. A negative resist can typically become less soluble in a developer upon irradiation, while a positive resist can typically become more soluble upon irradiation. In particular instances, resist material 36 can be a material is imageable by an electron beam and can be referred to as an e-beam resist.

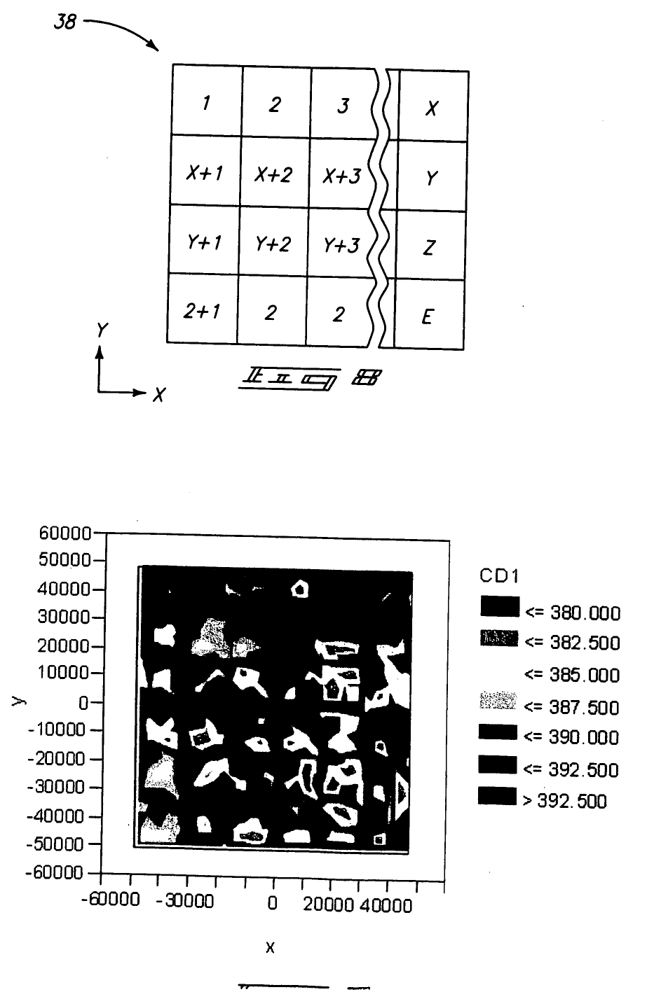

The substrate of FIG. 3 is shown to comprise a main-field region 38 and a boundary region 40 around the main-field region. A dashed border 39 is provided to demarcate a lateral periphery of the main-field located at a boundary between the main-field region and the boundary region. Persons of ordinary skill in the art will recognize that there is a main-field region of a reticle which can be defined as a region where openings will ultimately be formed for generating a feature pattern within a radiation-imagable material during a subsequent fabrication process (i.e. semiconductor fabrication processing) and that such main-field region will be spaced from edges of the reticle by a region which is not utilized to generate element patterns within the radiation-imagable material. Persons of ordinary skill in the art will also recognize that the spacing between the main-field region and the edge of the reticle is a boundary region and that such boundary region will typically extend entirely around the main-field region as is diagrammatically illustrated in FIGS. 3 and 4.

Material 36 comprises an upper surface, and FIG. 4 shows a view of preform 30 along such upper surface (i.e. shows a top view of the FIG. 3 substrate). The FIG. 4 view shows main-field region 38 having a substantially square lateral periphery defined by demarcation line 39, and additionally depicts boundary region 40 entirely surrounding the lateral periphery of the main-field region. Although the shown main-field region is square it is to be understood that the lateral periphery of the main-field region can have any suitable shape. Similarly, preform 30 can also have alternative shapes relative to that depicted in FIGS. 3 and 4. The methodology and concepts of the invention can be adapted for any appropriate reticle shape.

A series of marks 42 are provided within boundary region 40 to illustrate exemplary locations where alignment marks can ultimately be formed. Such alignment marks can be utilized for aligning masks during fabrication of the reticle as well as, or alternatively, for aligning the reticle during utilization of the reticle during patterning of light in a semiconductor fabrication process.

With reference to FIG. 5, preform 30 can be provided to a pattern writing system 50. System 50 can, in particular aspects, be a lithography system such as, for instance, an electron beam lithography system. An exemplary lithography system which can be utilized in accordance with the invention or which can be adapted for processing in accordance with the invention is the NUFLARE® Technology (Toshiba Kikai Kabushiki Kaisha Corp. Japan) EBM4000 electron beam lithography system.

Lithography system 50 can comprise an electron beam source 52 and a stage 54 for supporting preform 30 during the pattern writing process. In particular applications, source 52 can be configured to supply an electron beam at an energy level of approximately 50 keV, although alternative powers can be utilized. Stage 54 can preferably be a moveable stage, the movement of which is controlled by a controller 56. In particular applications, electron source 52 can additionally be controlled by controller 56. Accordingly, controller 56 can be utilized to control aspects of the electron beam emitted by source 52, for example, to provide or control a particular beam shape and/or intensity. In particular applications it can be desirable that beam source 52 provide an electron beam substantially perpendicular relative to the stage and/or upper surface of the preform.

Control of stage 54 can be utilized to scan the main-field of preform 30 in a particular exposure sequence. Referring again to FIG. 4, conventional e-beam exposure and scanning of a preform such as preform 30 is typically initiated at a corner of the main-field (e.g. the lower left hand corner), and proceeds horizontally (from left to right) to an opposing corner of the main-field. The scanning beam is then repositioned along the left hand border of the main-field at a position displaced or “stepped” vertically upward from the initial exposure point by a “frame height”. Such scanning continues continuously from left to right and from bottom to top until conclusion of the exposure at the top right hand corner of the main-field. Controller 56 (shown in FIG. 5) can be configured to control movement of moveable stage 54 to produce the described conventional scan sequence. As described below, in particular aspects of the invention, controller 56 can be configured to control stage 54 to provide an alternative scan pattern.

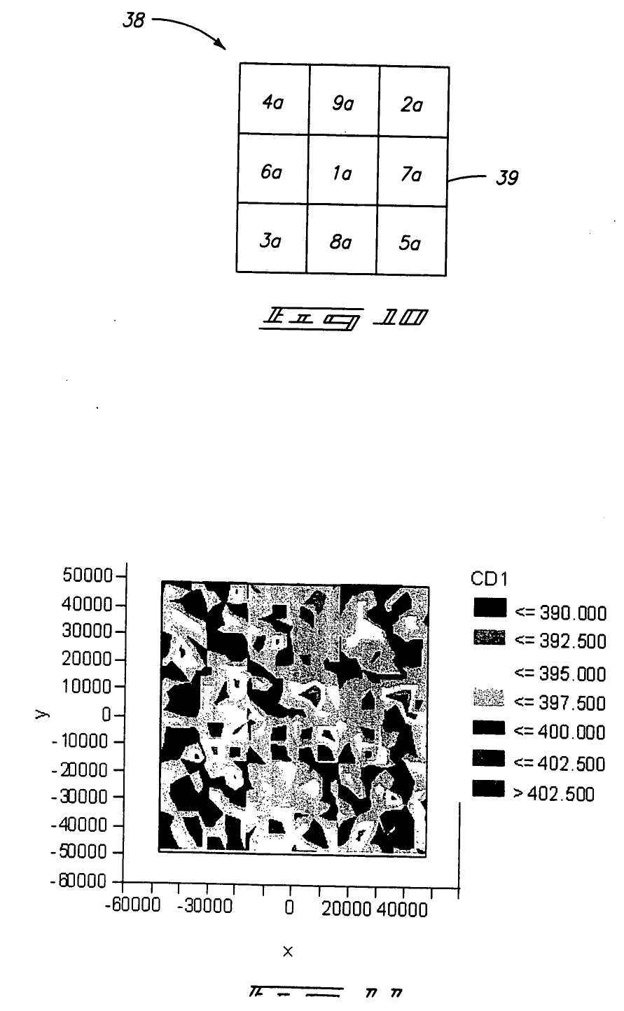

The above-described left to right, bottom to top scan sequence is alternatively described with reference to FIG. 6. As shown, main-field 38 can be divided into a plurality of exposure regions or areas (designated 1 through E). Such regions can be aligned in rows and columns to form a grid. The grid regions depicted in FIG. 6 are labeled with individual identifiers in accordance with the scan or exposure order or series described above (left to right, bottom to top). Main-field 38 as shown in FIG. 6 has also been assigned x and y directions as shown. For purposes of the description, the direction designated x can be referred to as a horizontal direction and the direction designated y can be referred to as a vertical direction.

In accordance with the described above described exposure sequence, e-beam exposure of the main-field would be initiated at region 1 and proceed horizontally in a continuous manner through region 2 and so on until exposure of region x has occurred. The exposure beam would then be stepped upwardly and scanning would resume at area x+1 and proceed horizontally in a continuous manner until region y has been exposed. Such left to right, bottom to top scanning would continue until final area E has been exposed and exposure is concluded. As further illustrated in FIG. 6 the number of columns present, between region 1 and region x for example, can vary. Although FIG. 6 shows four rows, it is to be understood that the number of rows can also vary. Accordingly, region 38 can be divided into a grid containing as many rows and columns as appropriate for a given main-field size and fragmentation of pattern data (discussed further below).

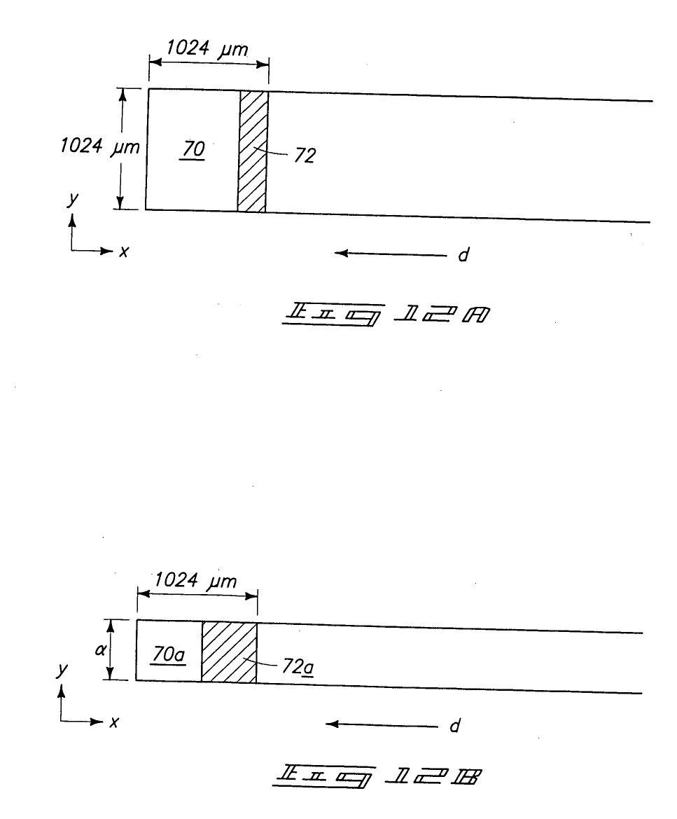

In a conventional e-beam patterning event which utilizes the scan strategy illustrated in FIG. 6 and application of a Gaussian dose correction, a critical dimension variation (or feature dimension variation) can be observed, as shown in FIG. 7. The critical dimension uniformity map shown in FIG. 7 is that of a 90 nm (1× half pitch) design rule gate layer reticle. The depicted CD distribution resulted from a beam scan on positive tone resist. As shown, a decrease in dark line CD (resist line, Cr line or MoSi line) is apparent near the top of the plate relative to that near the bottom of the plate. However, a similar difference from left to right is not apparent.

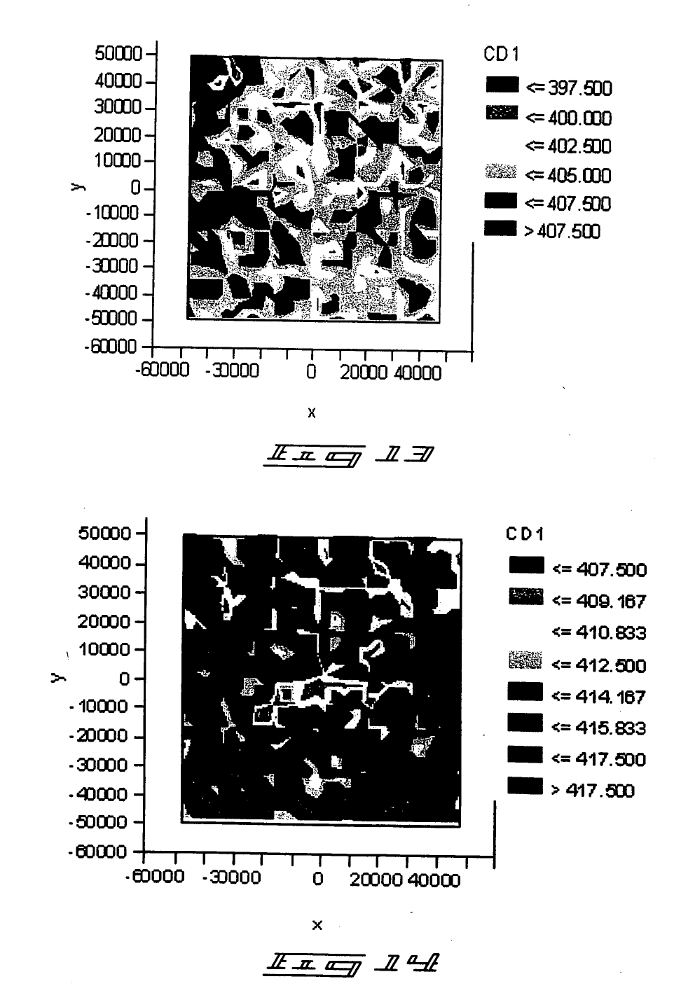

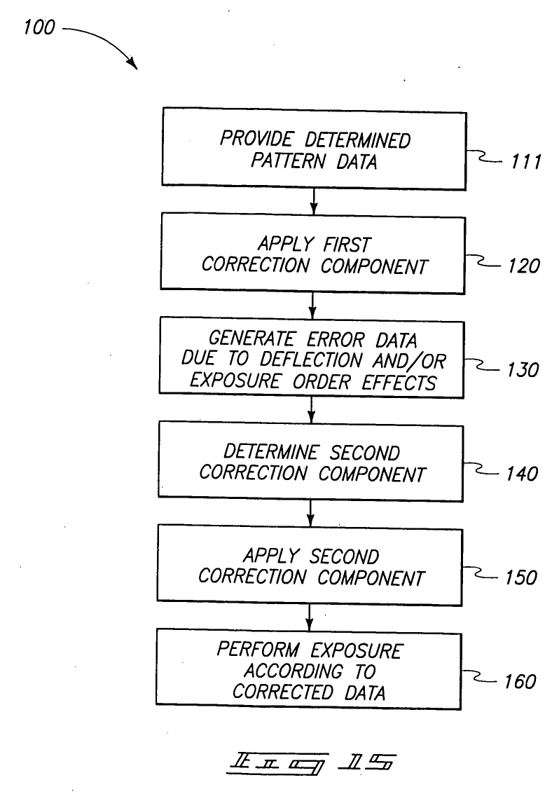

Referring next to FIG. 8, such shows an alternative scan order which was utilized to investigate the CD variation observed in FIG. 7. In the alternative scan sequence shown in FIG. 8, beam exposure was initiated at the top left corner of the main-field (grid region 1) and proceeded continuously from left to right until exposure of region x. The e-beam was displaced downwardly (in the −y direction) and the main-field was again scanned continuously from left to right from area x+1 to area y. Such left to right, top to bottom scanning was continued until concluding upon the completion of exposure of region E. The resulting CD distribution is shown in FIG. 9.

As shown, a decrease in dark line CD areas is observed near the bottom part of the plate. Similar left to right CD variation is not observed. The observation of decreasing dark line CD approaching the last row scanned indicates that the scan direction affects the fogging effect. Although the inventors are not intended to be held to any particular theory, it is proposed that the observed phenomenon could be the result of either or both of two possible causes. First, there is less fogging electron accumulation during the beginning portion of the chip exposure relative to the accumulation that results near the end of the exposure sequence. Accordingly, more fogging effect would occur at the end of the scanning process. Second, during the pattern exposure the resist heating effect hardens the adjacent resist and reduces its sensitivity to electrons. This hardening results in more effect of the electrons on ‘unexposed’ resist areas than to areas which have already been exposed during the scanning processing. This theoretical effect would also correlate to a stronger fogging effect near the end of a scan processing relative to that observed near the beginning of the scanning sequence. Accordingly, it could be advantageous to provide an alternative exposure initiation point with the pattern and/or an alternative scanning direction and sequence.

Referring to FIG. 10, an alternative main-field scanning strategy is described in accordance with the present invention. Main-field 38 is again divided into a plurality of grid regions or areas designated as areas 1a-9a. It is to be understood that the dividing of the main-field into a plurality of regions can comprise dividing into any appropriate number of grid regions. Further, it is to be understood that the grid regions can have alternative shapes relative to those shown in FIG. 10. Preferably the grid regions are all equivalent in area and are aligned in rows and columns as illustrated.

The plurality of grid regions shown in FIG. 10 can be described as comprising two outer rows of regions, a first of the two outer rows consisting of regions 4a, 9a and 2a, and a second of the two outer rows consisting of regions 3a, 8a and 5a. The plurality of grid regions further comprises two outer columns, a first outer column consisting of areas 4a, 6a and 3a, and a second outer column consisting of regions 2a, 7a and 5a. The plurality further comprises an inner or internal region 1a which is spatially separated from the boundary region or lateral periphery 39 of main-field 38 around the entirety of the main-field by at least one outer region.

Where main-field 38 is divided into a larger number of areas (rows and/or columns) than shown in FIG. 10, the main-field can comprise a plurality of inner areas and a number of outer areas which exceeds the 8 outer areas depicted in FIG. 10. For example, where main-field 38 is divided into 4 rows and 4 columns of regions, the resulting grid can contain 4 inner regions and 12 outer regions. A larger number of rows and/or columns can additionally increase the number of inner regions and outer regions. In some applications, it can be preferable to expose each of the internal regions comprised by a given grid prior to exposing any regions comprised by the outermost rows and columns.

The alternative scan order and scan initiation within main-field 38 as shown in FIG. 10 can be described as follows. Exposure to an electron beam can be initiated at an initial locus within internal area la. Scanning can proceed to a first corner region 2a followed by exposure of a second corner region 3a which is diagonally opposed relative to first corner 2a Exposure can subsequently proceed to a third corner 4a followed by exposure of a fourth corner 5a diagonally opposed relative to third corner 4a. Additional exposure is subsequently performed to expose sequentially the four non-corner regions 6a, 7a, 8a and 9a.

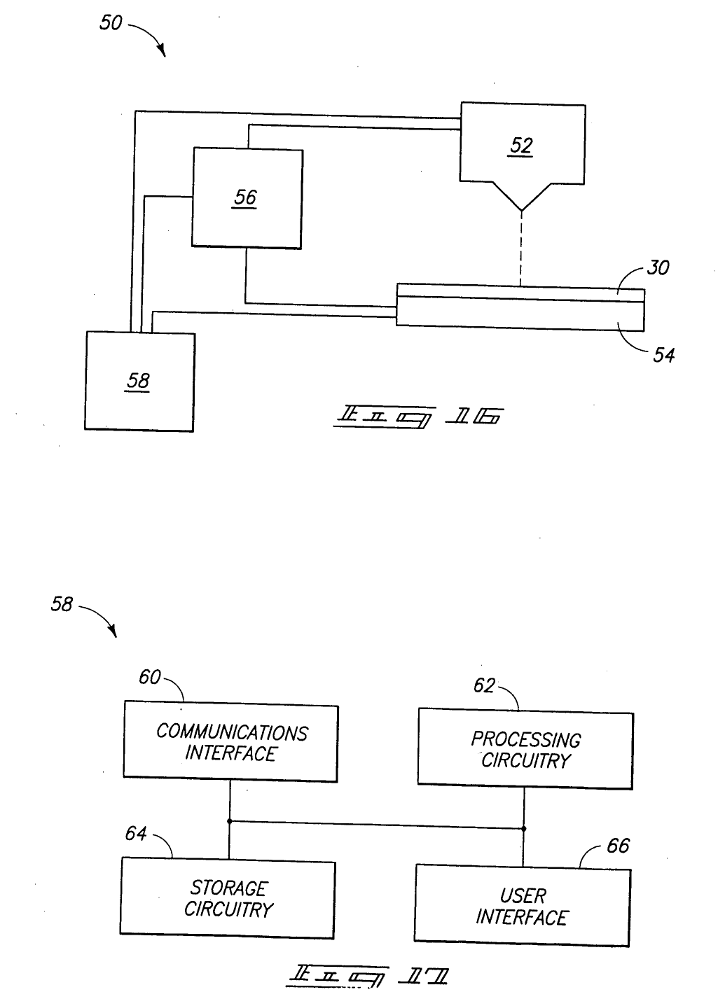

The observed CD distribution as a result of the alternative scan sequence depicted in FIG. 10 is shown in FIG. 11. E-beam scanning was conducted with an applied Gaussian dose correction as described above. The resulting critical dimension map is substantially more uniform relative to those obtained utilizing the left-to right, bottom to top scan strategy (FIG. 7) and the left to right, top to bottom scan strategy (FIG. 9). Accordingly, the alternative write strategy (exposure order) can distribute the fogging electrons more evenly allowing increased CD global uniformity across the main-field.

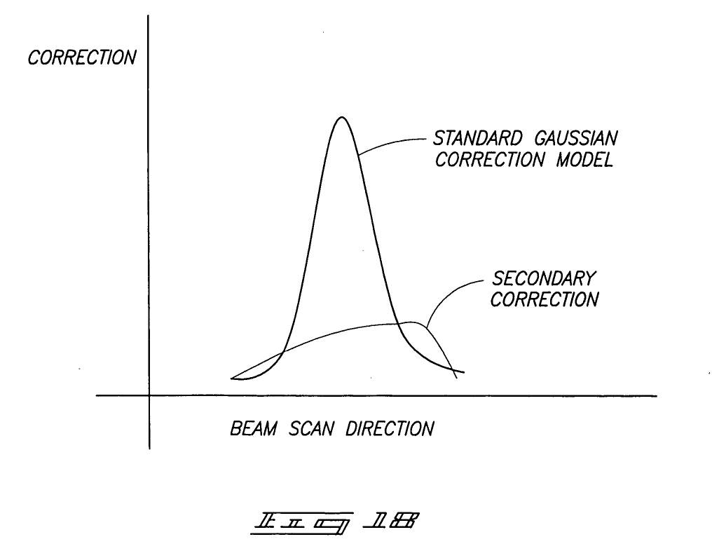

Another aspect of the invention is described with reference to FIG. 12. Panel A schematically illustrates an exemplary main deflection field 70 for a particular patterning protocol. Main deflection field 70 is shown to have a field area of 1024 microns×1024 microns. During a scanning process the moveable stage (shown in FIG. 5) moves in direction d (from right to left) such that a blank is scanned from left to right (horizontally in direction x) to produce the main deflection field 70. The field or frame height illustrated (1024 microns in the y direction) is determined by fragmentation of data into stripe fields during preparation of a patterning/exposure protocol. The deflection field width is affected by scan speed produced by stage movement in direction d. At typical write speeds, and where the main deflection field is square or substantially square such as shown in panel A, the exposure beam is deflected into the shaded area 72 most of the time. Accordingly, more fogging electron distribution occurs in the vertical direction (y) than in the horizontal direction (x).

Referring to panel B, an alternative main deflection field 70a can be produced in accordance with methodology of the present invention. Main deflection field 70a has a horizontal width of 1024 microns and a vertical height alpha (a) which is less than the deflection field width. Deflection field height (or frame height) α can be produced by alternative fragmentation (also referred to as fractionation) of data relative to a typical fragmentation utilized to produce the protocol illustrated in panel A. The height a is not limited to a particular value and can be, for example, from about 1 micron to about 1024 microns, and in particular instances can be from about 256 microns to about 768 microns, for applications where the main deflection field width is 1024 microns. Where the frame width is other than the exemplary 1024 microns as shown, α can have any value less than the frame width. In particular applications, α can preferably have a value such that the frame width is some integer multiple of α. More preferably, α has a value determined to produce a balanced deflection beam signature, where fogging distribution due to beam deflection is equivalent or substantially equivalent in the horizontal direction (stage scan direction) and vertical direction (orthogonal to the stage scan direction).

As shown in Panel B, due to a decreased frame height relative to that shown in panel A, the primary beam deflection area 72a has a greater width to height ratio (x/y) than would occur for larger stripe/frame height, such as where the value of α approaches the frame width. Accordingly, the shape of the primary deflection area 72a approaches a square shape dependent upon the speed of stage movement in direction d. The result is a more equivalent fogging effect distribution in the vertical and horizontal direction relative to the data fracturing and scan protocol depicted in panel A.

It can be advantageous to fragment data to produce a frame height a which is less than the frame width to minimize or eliminate the difference in fogging effect in the vertical relative to horizontal direction. The alternative data fragmentation and write protocol in accordance with the present invention as shown in Panel B can produce a more symmetrical fogging effect relative to the fogging effect which occurs using the protocol depicted in FIG. 12a. Accordingly, the conventional symmetrical (Gaussian) correction could more accurately correct CD variation throughout the main-field. The described alternative data fragmentation can therefore be utilized to produce reticles having increased global CD uniformity.

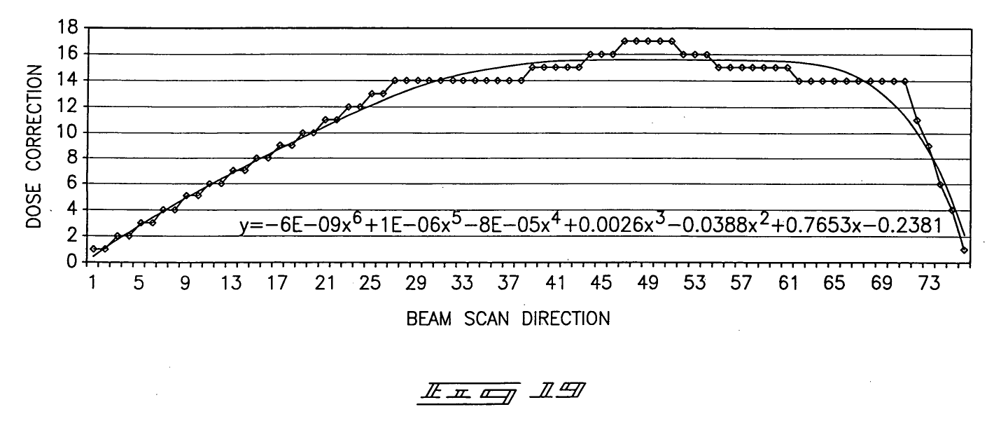

A critical dimension uniformity map of a reticle formed with a writing direction from bottom to top (as shown in FIG. 6) and a frame height of 512 microns is shown in FIG. 13. The decrease in dark line areas within the map near the right hand side indicates a possibility of increased fogging electron distribution in the horizontal direction as a result of the increased stage speed. A map of the critical dimension uniformity for a reticle processed according to a protocol having a bottom to top write direction (in accordance with FIG. 6) and a frame height of 768 microns is shown in FIG. 14. The increased uniformity observed suggests more uniform fogging electron distribution in the vertical and horizontal directions indicative of a balance between beam scan height and stage speed. The radial distribution signature may be minimized with an optimized symmetrical fogging effect correction.

An additional aspect of the invention is described generally with reference to FIG. 15. This aspect of the invention introduces methodology for providing a fogging effect correction which includes a correction component that at least partially corrects electron fogging contributed to by one or both of beam deflection and scan direction (exposure order). Preferably, the correction component can be added to a standard Gaussian correction component to produce an enhanced correction for application during a write protocol. Accordingly, the standard Gaussian correction can be referred to as a primary correction component and the added component can be referred to as a secondary correction component. Typically, the secondary correction component will be non-symmetrical due to non-symmetry of the fogging effects in the vertical and horizontal directions.

FIG. 15 shows processing 100 for enhancing critical dimension uniformity in accordance with the invention. At an initial stage 111, initial data can be provided pertaining to a predetermined or intended pattern. The initial pattern data can initially define pattern elements or features desired to be present in the final reticle construction. Additional processing 120 can apply a first correction component to the initial pattern data. This first correction component can be, for example, a symmetrical correction such as the Gaussian distribution correction model discussed above, and can be referred to as a primary correction component. Preferably, the primary correction component applied in process 120 at least partially alleviates a symmetrical component of critical dimension variation due to backscattering effects

An error determination process 130 can be performed to generate error data caused by beam deflection and/or exposure order effects as discussed above. A second correction component can be determined in a processing event 140 for alleviation of non-symmetrical feature dimension error based on the error data generated regarding deflection and/or exposure order effects. Processing events 130 and 140 can comprise generation of error data and determination of a second correction component utilizing empirical derivation or calculation based upon accumulated fogging effect in the beam scan direction.

In an additional processing event 150 the determined second correction component can be applied to the predetermined pattern data. Preferably, the second correction component is utilized as a secondary component and is applied in addition to the primary correction component. More preferably, the secondary correction component is added to the primary correction component to produce an overall correction which is applied to the determined pattern data to produce a corrected pattern utilizing the corrected pattern data.

Processing sequence 100 can further comprise exposure processing 160 where exposure of a reticle blank to an electron beam is performed in accordance with the corrected data. During processing event 160, exposing a reticle blank in accordance with corrected data can comprise utilizing dose correction, however it is to be understood that the invention additionally contemplates combining dose correction and beam shape variation techniques.

The exposed reticle blank can undergo further processing including developing the exposed resist and subsequent etching to eventually result in a reticle having enhanced global critical dimension uniformity relative to reticles prepared utilizing only the primary correction component.

In addition to the above processing sequence where error generation data processing 130 is performed after application of first correction component in step, the invention also contemplates obtaining error data in an absence of a first correction component. In other words, processing 140 can be performed to determine a correction component which is inclusive of back-scatter and deflection and/or scan order corrections.

The overall methodology depicted in FIG. 15 can be combined with alternative exposure initiation and exposure order aspects discussed above and/or alternative data fractionation and frame height aspects presented above. In this regard, processing 130 can comprise generation of error data where the data is generated utilizing a scan order and/or frame height which will be utilized during processing event 160.

A system for performing methodology in accordance with the invention is exemplified in FIG. 16. Lithography system 50 can include components similar or identical to those discussed above with reference to FIG. 5. System 50 can further comprise a processor 58 which can be configured to communicate with external devices including controller 56, moveable stage 54 and electron source apparatus 52. Although FIG. 16 depicts the components of lithography system 50 as being individual and independent components, it is to be understood that the invention contemplates integration of processor 58 and controller 56 within a single unit such as, for example, a computer.

Referring to FIG. 17, an exemplary configuration of processor 58 is shown. The depicted processor 58 includes a communications interface 60, processing circuitry 62, storage circuitry 64 and a user interface 66. Other circuitry or components can be provided in other embodiments and corresponding to the respective implementation or configuration of processor 58.

Communications interface 60 is configured to implement communications with respect to one or more device external of the computer, such as, for example stage 54, source 52 and/or controller 56. Communications interface 60 may comprise a wired or wireless connection to implement unidirectional or bidirectional communications in exemplary embodiments.

Processing circuitry 62 may execute executable instructions stored within articles of manufacture, such as memory, mass storage devices (e.g., hard disk drives, floppy disks, optical disks, etc.) or within another appropriate device, and embodied as, for example, software and/or firmware instructions.

In one embodiment, processing circuitry 62 is arranged to process data, control data access and storage, issue commands, and control other desired operations. Processing circuitry may comprise circuitry configured to implement desired programming provided by appropriate media in at least one embodiment. For example, the processing circuitry may be implemented as one or more of a processor and/or other structure configured to execute executable instructions including, for example, software and/or firmware instructions, and/or hardware circuitry. Exemplary embodiments of processing circuitry include hardware logic, PGA, FPGA, ASIC, state machines, and/or other structures alone or in combination with a processor. These examples of processing circuitry are for illustration and other configurations are possible.

Storage circuitry 64 is configured to store electronic data and/or programming such as executable instructions (e.g., software and/or firmware), data, or other digital information and may include processor-usable media. Processor-usable media includes any article of manufacture which can contain, store, or maintain programming, data and/or digital information for use by or in connection with an instruction execution system including processing circuitry in the exemplary embodiment. For example, exemplary processor-usable media may include any one of physical media such as electronic, magnetic, optical, electromagnetic, infrared or semiconductor media. Some more specific examples of processor-usable media include, but are not limited to, a portable magnetic computer diskette, such as a floppy diskette, zip disk, hard drive, random access memory, read only memory, flash memory, cache memory, and/or other configurations capable of storing programming, data, or other digital information.

User interface 66 is configured to interact with a user including conveying data to a user (e.g., displaying data for observation by the user, audibly communicating data to a user, etc.) as well as receiving inputs from the user (e.g., tactile input voice instruction, etc.). Accordingly, in one exemplary embodiment, the user interface may include a display (e.g., cathode ray tube, LCD, etc.) configured to depict visual information and an audio system as well as a keyboard, mouse and/or other input device. Any other suitable apparatus for interacting with a user may also be utilized.

In one embodiment the processing circuitry 62 can comprise circuitry configured to implement desired programming in accordance with methodology of the invention. For example, the processing circuitry may be implemented to perform processing in accordance with FIG. 15. Accordingly, the invention encompasses recording media comprising programming configured to cause processing circuitry to perform processing comprising one or more of the processing events depicted in FIG. 15 (or alternative processing described above), and additionally encompasses lithography systems such as that shown in FIG. 16 containing such recording medium.

A recording medium in accordance with the invention can comprise programming configured to cause processing circuitry to access data defining a design pattern to be written onto a resist material by exposure to an exposure beam such as, for example, an electron beam. The programming can additionally be configured to cause processing circuitry to obtain error data pertaining to feature dimension variation in a resulting exposure pattern relative to the initial design pattern. In particular aspects, the obtaining error data can be performed after application of a primary correction component such as the symmetrical distribution component discussed above. Accordingly, the obtained error data can correspond to feature dimension variation caused by exposure beam deflection during writing of the design pattern and/or dimension error caused by fogging due to effects of write order.

The recording medium can further comprise programming configured to cause processing circuitry to generate correction data based on the error data. Further, the programming can be configured to cause the processing circuitry to produce data defining a corrected pattern by adjusting the initial data defining the design pattern utilizing the correction data. Programming can additionally be configured to cause processing circuitry to apply the corrected pattern during an exposure event.

Obtaining error data pertaining to a feature dimension variation and an exposure pattern can comprise producing one or more test reticles and/or can comprise user input. Preferably the obtaining error data comprises obtaining data utilizing an exposure initiation site, exposure order and frame height which will be utilized during applying the corrected pattern in an exposure event.

Referring to FIG. 18, such depicts an enhanced correction model in accordance with methodology of the invention. As shown, a standard Gaussian correction model can be applied as a primary correction component. A secondary correction component such as the exemplary secondary correction component depicted in FIG. 18 can be applied preferably by adding the secondary correction component to the primary correction component to produce an overall correction to be applied during writing of a reticle.

As indicated above, the secondary correction component can be determined by writing one or more test plates (reticles). The test plates can then be utilized to produce a feature uniformity map and/or a dose correction map utilizing the average data. The resulting determined correction can then be applied to a write pattern to enhance correction relative to a reticle formed utilizing only the primary correction component. Alternatively, the secondary electron fogging correction can be calculated by, for example, applying a polynomial correction equation. By way of example, a six order polynomial correction y=a+bx+cx²+dx³+ex⁴+fx⁵+gx⁶ can be applied where y is the dose correction and x corresponds to a dominant fogging distribution direction. The constants a through g can be adjusted for slope and magnitude. Exemplary 6 order polynomial correction models are shown in FIGS. 19 and 20. In such figures, the connected point's curve corresponds to the raw data while the smooth curve corresponds to the simulation.

The secondary electron fogging correction may alternatively be calculated theoretically based on the pattern density, the x, y main deflection field dimension, the stage speed and the strength of the fogging effect upon the unexposed resist relative to the fogging effects upon exposed resist. The calculated result would be similar to the polynomial correction described above.

Application of the methodologies of the present invention during reticle writing can produce reticles having enhanced global uniformity relative to conventional techniques. The enhanced global uniformity of the reticle can in turn minimize or eliminate CD variation during subsequent patterning of, for example, a semiconductor wafer.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A method of forming a mask pattern comprising:
providing a mask blank having a metallic layer disposed between a transparent substrate material and an imageable material, the mask blank comprising an upper surface having a defined main-field area bounded by a lateral periphery;
defining a plurality of regions of substantially equivalent area within the main-field, the plurality of regions comprising an inner region and multiple outer regions, the inner region being spaced from the entirety of the lateral periphery by at least one outer region; and
initiating exposure of the mask blank to an electron beam at an initial locus within the inner region.

2. The method of claim 1 wherein the main-field consists of nine regions arranged in three rows and three columns.

3. The method of claim 2 wherein the nine regions comprise a central region and four corner regions, the four corner regions comprising a first corner region, a second corner region diagonally opposed to the first corner region, a third corner region, and a fourth corner region diagonally opposed to the third corner region, and wherein writing of the unit pattern is initiated at the central region and proceeds from the central region to the first corner region, followed by the second corner region, followed by the third corner region followed by the fourth corner region and subsequent exposure of four remaining non-corner regions.

4. The method of claim 1 wherein the main-field consists of 16 regions arranged in four rows and four columns, wherein twelve regions are outer regions and four regions are inner regions, and wherein initiation of exposure occurs within one of the four inner regions.

5. The method of claim 4 wherein each of the four inner regions is exposed prior to exposing any of the twelve outer regions.

6. A method of forming a mask pattern comprising:
determining pattern writing data for a given exposure pattern;
fractionating the data into stripe fields having a stripe height;
providing a mask blank within an exposure apparatus; and
exposing the mask blank to a charged beam, the exposing comprising scanning at a constant speed in a horizontal direction, the scan speed creating a beam deflection having a vertical deflection beam height defined by the stripe height and a horizontal deflection beam width substantially equivalent to the beam height.

7. The method of claim 6 wherein the stripe fields have a stripe length at least about twice the stripe height.

8. The method of claim 7 wherein the stripe length is 1024 microns and the stripe height is 512 microns.

9. The method of claim 7 wherein the stripe length is 1024 microns and the stripe height is 768 microns.

10. The method of claim 7 wherein the stripe length is 1024 microns and the stripe height is 256 microns.

11. A method of correcting feature dimension variation comprising:
subjecting a mask blank to an initial write pattern exposure to form an initially exposed substrate, the initial write pattern having a first dose correction component that is symmetrical relative to a center of the initial write pattern;
dividing the substrate into a plurality of regions across an upper mask surface;
measuring the feature dimension within of each region and determining the variance between the measured feature dimension and an intended feature dimension;
utilizing the variance to determine a second dose correction component;
creating an enhanced dose correction comprising the second dose correction component added to the first dose correction component to at least partially correct the variance;
applying the enhanced dose correction to a write pattern to generate an enhanced correction write pattern; and
exposing a subsequent mask blank in accordance with the enhanced dose correction write pattern.

12. The method of claim 11 wherein the second correction component is non-symmetrical relative to the center of the initial write pattern.

13. The method of claim 11 wherein the first correction component alleviates dimension variance caused by backscatter of an exposure beam and the second correction component at least partially alleviates variance caused by deflection of the exposure beam.

14. A microlithography method comprising:
providing a substrate comprising an upper surface having an exposure region;
dividing the exposure region into a plurality of grid regions;
defining a scan initiation point and scan sequence for exposing the plurality of grid regions to a charged beam; and
determining an exposure dose correction component for the plurality of grid regions dependent upon the position of each grid region within the exposure region relative to the scan initiation point and dependent upon scan sequence.

15. The method of claim 14 wherein the exposure dose correction component is a first component comprised by an overall dose correction, and wherein the first component is added to a second component, the second component being a Gaussian distribution dose correction.

16. The method of claim 14 wherein the exposure dose correction component is a first component comprised by an overall dose correction, and wherein the first component is added to a second component, the second component adjusting dosage to compensate for back-scattering effects and the first component adjusting dosage to compensate at least one of scan direction effects and beam deflection effects.

17. The method of claim 14 wherein the exposure dose correction component is non-symmetrical.

18. The method of claim 14 wherein the plurality of grid regions are aligned within at least three rows and at least three columns, there being two outermost rows, two outermost columns and at least one interior grid region not comprised by the two outermost rows and two outermost columns.

19. The method of claim 18 wherein the scan initiation point is within an interior grid region.

20. The method of claim 18 wherein the scan initiation point is within a corner grid region disposed at an intersection of an outer row and an outer column.

21. The method of claim 20 wherein the corner grid region is a first corner grid region and wherein the scan sequence terminates at a second corner grid region diagonally disposed relative to the first corner grid region.

22. The method of claim 18 wherein all interior grid regions are exposed prior to exposing any grid region comprised by the two outer rows and two outer columns.

23. The method of claim 14 further comprising providing write data to a charged-beam exposure apparatus, the write data being fractionated into stripes having a stripe height such that beam deflection in a beam scan direction is substantially equivalent to beam deflection in a second direction, the second direction being orthogonal relative to the scan direction.

24. A recording medium comprising programming configured to cause processing circuitry to perform processing comprising:
accessing data defining a design pattern to be written on a resist material by exposing to an exposure beam;
obtaining error data pertaining to feature dimension variation in a resulting exposure pattern relative to the design pattern, the feature dimension variation being caused by exposure beam deflection during writing of the design pattern;
generating correction data based upon the error data;
producing data defining a corrected pattern by adjusting the data defining the design pattern utilizing the correction data; and
applying the corrected pattern during an exposure event.

25. The recording medium of claim 24 wherein data defining the design pattern comprises a first correction component that alleviates feature dimension variation caused by backscattering, and wherein the producing a corrected pattern comprises combining the first correction component with a second correction component, the second correction component comprising the correction data generated based upon the error data.

26. The recording medium of claim 25 wherein the first correction component is based upon-a standard deviation relative to a reference point within the design pattern.

27. The recording medium of claim 25 wherein the second correction component is a distribution based upon an exposure position relative to an exposure initiation point for the exposure pattern.

28. The recording medium of claim 24 wherein the programming is further configured to cause processing circuitry to apply the corrected pattern data during a mask patterning process.

29. An electron beam exposure system comprising:
an electron beam source;
a movable stage for supporting a substrate;
a processor comprising programming configured to cause processing circuitry to perform processing events comprising:
accessing exposure pattern data;
dividing the exposure pattern into regions;
determining a dose correction for each region, the dose correction comprising a first component to alleviate backscattering effects and a second component to alleviate beam deflection effects; and
generate a corrected exposure pattern to apply a corrected dose for each region; and
a controller being configured to direct electrons emitted by the electron beam source to expose a layer of resist on a mask blank in accordance with the corrected exposure pattern, the directing electrons comprising moving the movable stage.

30. The system of claim 29 wherein the exposure of the resist is performed at an energy level of approximately 50 keV.
